(19) United States
(12) Patent Application Publication
(10) Pub. No.: US 2007/0213382 A1
(43) Pub. Date: Sep. 13, 2007

(54) 2-METHYLTHIAZOLIDINE-2, 4-DICARBOXYLIC ACID-CONTAINING COMBINATION PREPARATIONS

(76) Inventors: Susilo Rudy, Koln (DE); Eberhard Amtmann, Heidelberg (DE)

Correspondence Address: J C PATENTS, INC., 4 VENTURE, SUITE 250, IRVINE, CA, 92618, US

(21) Appl. No.: 11/568,506

(22) PCT Filed: May 2, 2005
(86) PCT No.: PCT/DE05/00810

§ 371 Date: Oct. 30, 2006

Related U.S. Application Data
(60) Provisional application No. 60/569,570, filed on May 10, 2004.

(30) Foreign Application Priority Data
May 3, 2004 (DE) 10-2004-021-658.4

(51) Int. Cl. A61K 31/426; A61K 38/19; A61K 38/09; A61K 38/16; A61K 38/18; A61K 31/704; A61K 31/7048; A61K 31/522; A61K 31/7072; A61K 31/513; A61K 31/4745

(52) U.S. Cl. 514/365; 514/34; 514/27; 514/492; 514/109; 514/8; 514/283; 514/12; 514/171; 514/449; 424/85.1; 424/649; 424/155.1; 424/94.63; 514/49; 514/263.34; 514/251; 514/269; 514/410; 514/15

(57) ABSTRACT

The invention relates to a combination preparation of 2-methylthiazolidine-2,4-dicarboxylic acid and/or physiologically acceptable salts thereof and at least one cytotoxic and/or cytostatic compound as well as the use of these combination preparations for the treatment of cancer.

The invention relates to a combination preparation of 2-methylthiazolidine-2,4-dicarboxylic acid and/or physiologically acceptable salts thereof and of at least one antiangiogenic cytotoxic and/or cytostatic compound. The invention relates further to the use of these combination preparations for the treatment of cancer as well as to the increase of the efficacy of antiangiogenic and/or cytotoxic and/or cytostatic compounds by the additional administration of 2-methylthiazolidine-2,4-dicarboxylic acid.

The synthesis of 2-methylthiazolidine-2,4-dicarboxylic acid is well known from DE-OS 21 16 629. EP 0 969 834 B1 discloses the use of 2-methylthiazolidine-2,4-dicarboxylic acid as mucolytic agent, EP 98 916 809 describes a combination preparation of 2-methylthiazolidine-2,4-dicarboxylic acid and paracetamol, and EP 01 915 023 discusses the use of 2-methylthiazolidine-2,4-dicarboxylic acid for the treatment of infective diseases, especially HIV.

European patent EP 1 255 538 B1 discloses the use of 2-methylthiazolidine-2,4-dicarboxylic acid for the treatment and prophylaxis of cancer. In this patent the use of 2-methylthiazolidine-2,4-dicarboxylic acid for the preparation of a drug for prevention and/or reduction of undesired side effects of cytostatics is also described.

Cytostatics represent substances which prevent or considerably delay the initiation and interrupt or respectively disturb the cycle of the nuclear and/or plasma division (karyokinesis or respectively cytokinesis). They interfere either with the reduplication or the transcription of the DNA or with the formation and disjunction of its carrier structures and lead to division perturbing chromosome aberrations or respectively suppress the formation and disturb the function of the spindle apparatus (and are almost always mutageneous). In the broader sense, cytostatics also represent substances which respectively disturb and prevent the provision of the necessary energy for the nucleoprotein synthesis or the spindle function. Generally, they are classified into antimetabolites, alkylating agents, cytostatically active antibiotics and mitosis inhibitors according to their mode of action (Roche Lexikon Medizin, 4. Edition; © Urban & Fischer Verlag, Munchen 1984/1987/1993/1999).

Paclitaxel (Taxol®) is probably one of the best investigated cytostatics.

EP 1 255 538 B1 discloses the use of 2-methylthiazolidine-2,4-dicarboxylic acid for the reduction of undesired side effects of cytostatics. Thus, EP 1 255 538 B1 gives a solution for the problem of reducing undesired side effects of cytostatics.

The object of the present invention, however, is to increase the effect, i.e. the efficacy and/or activity of cytostatics.

This object is solved by the technical teaching of the independent patent claims. Advantageous embodiments of the invention are given in the dependent patent claims, the description as well as in the examples.

Surprisingly, it was shown that the above described effect of cytostatics can be increased by administration of 2-methylthiazolidine-2,4-dicarboxylic acid.

Thus, the present invention relates to combination preparations which comprise 2-methylthiazolidine-2,4-dicarboxylic acid and/or physiologically acceptable salts thereof and at least one antiangiogenically and/or cytotoxically and/or cytostatically active compound.

2-Methylthiazolidine-2,4-dicarboxylic acid is a chemical compound with two stereogenic centers wherein the R-configuration at position 4 is especially preferred. The stereogenic centre at position 2 has no preferred configuration.

The term “2-methylthiazolidine-2,4-dicarboxylic acid” is supposed to refer to the diastereomeric compounds (2R,4R)-2-methylthiazolidine-2,4-dicarboxylic acid, (2S,4R)-2-methylthiazolidine-2,4-dicarboxylic acid as well as (2RS,4R)-2-methyl-thiazolidine-2,4-dicarboxylic acid. In the case of (2RS,4R)-2-methylthiazolidine-2,4-dicarboxylic acid, the molar ratio of (2R,4R)-2-methylthiazolidine-2,4-dicarboxylic acid to (2S,4R)-2-methylthiazolidine-2,4-dicarboxylic acid can range from 90:10 to 10:90, wherein the percentages of (2R,4R)-2-methylthiazolidine-2,4-dicarboxylic acid preferably prevails.

Furthermore, the term “2-methylthiazolidine-2,4-dicarboxylic acid” is supposed to comprise not only the free acid but also physiologically acceptable salts. Accordingly, the compound 2-methylthiazolidine-2,4-dicarboxylic acid and respectively an enantiomer, diastereomer or an enantiomeric and/or diastereomeric mixture thereof can be administered as free acid and/or in form of the pharmacologically acceptable salt. Suitable examples of these salts comprise acid addition salts and alkaline metal salts. Thus, alkaline metal salts can be used, such as the sodium salt, the potassium salt, the lithium salt, the magnesium salt, the calcium salt and/or alkyl ammonium salts. As acids which form an acid addition salt the following ones can be mentioned: sulphuric acid, sulphonic acid, phosphoric acid, nitric acid, nitrous acid, perchloric acid, hydrobromic acid, hydrochloric acid, formic acid, acetic acid, propionic acid, succinic acid, oxalic acid, gluconic acid (glyconic acid, dextronic acid), lactic acid, malic acid, tartaric acid, tartronic acid (hydroxymalonic acid, hydroxypropionic diacid), fumaric acid, citric acid, ascorbic acid, maleic acid, malonic acid, hydroxymaleic acid, pyruvic acid, phenylacetic acid, (o, m, p)-toluic acid, benzoic acid, p-aminobenzoic acid, p-hydroxybenzoic acid, salicylic acid, p-aminosalicylic acid, methanesulfonic acid, ethanesulfonic acid, hydroxyethanesulfonic acid, ethylenesulfonic acid, p-toluenesulfonic acid, naphthylsulfonic acid, naphthylamine sulfonic acid, sulfanilic acid, camphersulfonic acid, china acid (canine acid), o-methylmandelic acid, hydrogen-benzenesulfonic acid, picric acid (2,4,6-trinitrophenol), adipic acid, and D-o-tolyltartaric acid. Amino acids can also be used for the salt formation such as glycine, alanine, valine, leucine, isoleucine, serine, threonine, phenylalanine, tyrosine, tryptophan, lysine, arginine, histidine, aspartic acid , glutamic acid, asparagine, glutamine, cysteine, methionine and proline wherein the amino acids methionine, tryptophan, lysine and arginine are preferred.

A combination preparation which contains 2-methylthiazolidine-2,4-dicarboxylic acid, i.e. at least one enantiomer or a diastereomer of 2-methylthiazolidine-2,4-dicarboxylic acid as free acid or as salt and at least one antiangiogenic and/or at least one cytotoxic compound and/or at least a cytostatic compound does not have to contain obligatorily both active agents in one pharmaceutical formulation.

Obviously, pharmaceutical formulations can be provided which contain 2-methylthiazolidine-2,4-dicarboxylic acid together with the antiangiogenic and/or cytotoxic and/or cytostatic compound. The preferred preparations, however, are combination preparations which comprise two separate pharmaceutical formulations, namely one for 2-methylthiazolidine-2,4-dicarboxylic acid and another one for the at least one antiangiogenic and/ or cytotoxic and/or cytostatic compound. This is advantageous since both active agents can be applied simultaneously but the administered amounts of both compounds as well as the specific time of application of both compounds can be varied. It is furthermore advantageous to administer the compound 2-methylthiazolidine-2,4-dicarboxylic acid which only leads to low side effects before, simultaneously or after the administration of the antiangiogenic and/ or cytotoxic and/or cytostatic compound. Before the administration of the cytotoxic and/or cytostatic compound may mean 6 h, 12 h, 18 h, 24 h, 30 h, 36 h, 42 h, 48 h, 54 h or 60 h before. The term “simultaneously” is supposed to refer to both, the administration of both active agents in a single formulation and an administration delayed to up to one hour of one active agent. After the administration of the antiangiogenic and/or cytotoxic and/or cytostatic compound can mean 6 h, 12 h, 18 h, 24 h, 30 h, 36 h, 42 h, 48 h, 54 h or 60 h thereafter. Preferred is an administration of 2-methylthiazolidine-2,4-dicarboxylic acid after the administration of the antiangiogenic and/or cytotoxic and/or cytostatic compound and especially preferred 24 h or 48 h thereafter.

Thus, the combination preparation comprises preferably a pharmaceutical formulation containing 2-methylthiazolidine-2,4-dicarboxylic acid and/or physiologically acceptable salts thereof as well as at least one other pharmaceutical formulation containing the at least one antiangiogenic and/or cytotoxic and/or cytostatic compound.

Among others, the following may be used as antiangiogenic and/or cytotoxic and/or cytostatic compounds, i.e. chemical compounds with antiangiogenic and/or cytotoxic and/or cytostatic properties: alkylating agents, antibiotics with cytostatic properties, antimetabolites, microtubule inhibitors and topoisomerase inhibitors, compounds containing platinum and other cytostatics (cytostatic and/or cytotoxic active agents) such as asparaginase, tretinoin, alkaloids, podophyllotoxins, taxanes and Miltefosine®.

The combination of 2-methylthiazolidine-2,4-dicarboxylic acid and/or physiologically acceptable salts thereof with other tumor therapeutic agents such as hormones, immunomodulators, monoclonal antibodies, signal transductors (signal transduction molecules) and cytokines is also applicable according to invention.

Examples for alkylating agents are, among others, chlorethamine, cyclophosphamide (cytoxan), trofosfamide, ifosfamide (Ifex), melphalan (Alkeran), mechlorethamine hydrochloride (Mustargen), chlorambucil (Leukeran), busulfan (Myleran), thiotepa, carmustine, cisplatine, (Platinol), carboplalomustine, dacarbazine, procarbazine, temozolomide, treosulfan, estramustine and nimustine.

Examples for antibiotics with cytostatic properties are daunorubicin as well as liposomal daunorubicin (Daunomycin, Cerubidine), doxorubicin (adriamycin) as well as liposomal adriamycin, dactinomycin, mitomycin C, bleomycin (Blenoxane), epirubicin (4-epi-adriamycin), idarubicin (Idamycin), dactinomycin (Actinomycin D, Cosmegen), mitoxantrone (Novantrone), mitomycin C (Mitomycin), Plicamycin (Mithracin), amsacrine and actinomycin D.

Methotrexate (MTX, Mexate), 5-fluorouracil (Fluoracil, 5-FU), 6-thioguanine (Thioguanine, 6-TG), 6-mercaptopurine, fludarabine (Fludara), floxuridine (FUDR), cladribine, pentostatin, gemcitabine (Gemzar), cytarabine, azathioprine, raltitrexed, capecitabine (Xeloda), cytosine arabinoside, deoxycoformycin (Pentostatin, Nipent), thioguanine and mercaptopurine (6-MP, Purinethol) can be mentioned as examples for antimetabolites (antimetabolitic active substances).

Accounted among the class of alkaloids and podophyllotoxins are for example vincristine, vinblastine, vindesine, etoposide as well as teniposide. Furthermore, compounds containing platinum can be used according to invention. As compounds containing platinum are to be mentioned for instance cisplatin, carboplatin and oxaliplatin. Accounted among the microtubule inhibitors are for example alkaloids such as vinca alkaloids (vincristine, vinblastine, vindesine, vinorelbine. Among the topoisomerase inhibitors are for instance etoposide (VP-16, Etopophos, VePesid) and teniposide (VM-26, Vumon) and among the camptothecins are topotecan (Hycamtin) and irinotecan (Camptosar) and among the nitrosourea compunds are carmustine (BCNU), lomustine (CCNU) and streptozocin (Zanosar). Possible topoisomerase inhibitors are for example, etoposide, teniposide, camptothecin, topotecan and irinotecan.

Paclitaxel and docetaxel are examples for the compound class of the taxanes and accounted among the other cytostatic active agents (other cytostatics) are for example hydroxycarbamide (hydroxyurea), imatinibe, Miltefosine®, amsacrine, topotecan (topoisomerase-1 inhibitor), pentostatin, bexarotene, tretinoin and asparaginase. Representatives of the compound class of the monoclonal antibodies are among others trastuzumab (also known as Herceptin®), alemtuzumab (also known as MabCampath®) and rituximab (also known as MabThera®).

According to invention, hormones such as glucocorticoids (prednisone), hydrocortisones, dexamethasones (Decadron), estrogens [fosfestrol, estramustine (Emcyt), chlorotrianisene (TACE), diethylstilbestrol (DES)], aromatase inhibitors [anastrozole (Arimidex)], LHRH (buserelin, goserelin, leuprorelin, triptorelin), flutamide (Eulexing), bicalutamide (Casodex), leuprolide (Lupron), goserelin acetate (Zoladex), cyproterone acetate, progestine (Medoxyprogesterone acetate [(Depo-provera)], megestrol acetate (Megacel), tamoxifen, toremifen, aminoglutethimide, formestane, exemestane, letrozole and anastrozole can also be used. Accounted among the classes of the immunomodulators, cytokines, antibodies and signal transductors are interleukin-2, interferon-α, erythropoietin, G-CSF, trastuzumab (Herceptin®), rituximab (MabThera®), gefitinib (Iressa®), ibritumomab (Zevalin®), levamisole as well as retinoids. Further active substances which can be used according to the invention are asparaginase (Elspar), pegaspargase (Oncaspar), hydroxyurea (Hydrea, Mylocel), procarbazine (Matulane) and imatinib mesylate (Gleevec).

Furthermore, combinations as well as the uses of combinations described herein are preferred, wherein these combinations consist of a cytostatic combined with 2-methylthiazolidine-2,4-dicarboxylic acid and or salts of 2-methylthiazolidine-2,4-dicarboxylic acid and a antiangiogenic active substance.

Among the antiangiogenic active substances are mainly counted such substances which interfere with or inhibit the formation of blood vessels. Therefore, antiangiogenic active substances are particularly those pharmacological active substances interfering with or inhibiting the arrangement, migration, proliferation and organisation of endothelium cells to a blood vessel. Antiangiogenic substances are particularly described as antiangiogenic if their antiangiogenic characteristics are proven by at least one testing procedure, for example the CAM assay.

Antiangiogenic active substances are for example classified according to their modes of action and are also described as angiogenesis inhibitors and modulators. The following antiangiogenic active substances classified according to their modes of action may be used according to the invention:
1. Inhibitors of the matrix decomposition (MMP inhibitors: Neovastat, plasmine inhibitors);
2. Inhibitors of the endothelium cell proliferation
   VEGF inhibitors: bevamizumab, avastin, angiozyme
   PDGF inhibitor: gleevec
   Plasminogene/collagene XVIII inhibitors: endostatin, angiostatin
   Antivascular substances: combrestatin
   Integrin inhibitors: vitaxin
3. Upstream modulators (trastuzumab, cetuximab)
4. Substances with unknown modes:
   Calcium channel blocker: carboxyamido thiazole;
   COX-2-inhibitors: celecoxib;
   Hypoxic substances: tirapazamine;
   NFkB modulators
   Thalidomide

Among the antiangiogenic substances are particularly counted the antimitotically acting substances, taxanes (paclitaxel, derivatives of paclitaxel (Taxol®)) and vinca alkaloids (vincristine, vinblastine), adriamycin, doxorubicin, idarubicin, 5-fluorouracil, etoposide, protamine, methotrexate, etretinate, dexamethasone and thalidomide (Contergan®, chemical formulation: (±)-N-(2,6-dioxo-3-piperidyl)phtalamide).

Thus, combinations of 2-methylthiazolidine-2,4-dicarboxylic acid with an antiangiogenic substance and an active substance selected from the group comprising chlorethamine, cyclophosphamide, trofosfamide, ifosfamide, melphalan, chlorambucil, busulfan, thiotepa, carmustine, lomustine, dacarbazine, procarbazine, temozolomide, treosulfan, estramustine, nimustine, daunorubicin as well as liposomal daunorubicin, doxorubicin (adriamycin) as well as liposomal adriamycin, dactinomycin, mitomycin C, bleomycin, epirubicin (4-epi-adriamycin), idarubicin, dactinomycin, mitoxantrone, plicamycin, actinomycin D, methotrexate, 5-fluorouracil, 6-thioguanine, 6-mercaptopurine, fludarabine, cladribine, pentostatin, gemcitabine, cytarabine, azathioprine, raltitrexed, capecitabine, cytosine arabinoside, thioguanine, mercaptopurine, vincristine, vinblastine, vindesine, cisplatin, carboplatin, oxaliplatin, vinca alkaloids, vinorelbine, etoposide, teniposide, camptothecin, topotecan, irinotecan, paclitaxel, docetaxel, hydroxycarbamide (hydroxyurea), imatinib, Miltefosine®, amsacrine, topotecan (topoisomerase-I inhibitor), pentostatin, bexarotene, tretinoin, asparaginase, trastuzumab, alemtuzumab, rituximab, glucocorticoids (prednisone), estrogens (fosfestrol, estramustine), LHRH (buserelin, goserelin, leuprorelin, triptorelin), flutamide, cyproterone acetate, tamoxifen, toremifen, aminoglutethimide, formestane, exemestane, letrozole, anastrozole, interleukin-2, interferon-α, erythropoietin, G-CSF, trastuzumab (Herceptin®), rituximab (MabThera®), gefitinib (Iressa®), ibritumomab (Zevalin®), levamisole, wherein cisplatin, temozolomide and vincristine are especially preferred.

The preferred antiangiogenic substance in these three-way combinations is paclitaxel. The following three-way combinations are especially preferred: 2-methylthiazolidine-2,4-dicarboxylic acid with paclitaxel and cisplatin; 2-methylthiazolidine-2,4-dicarboxylic acid with paclitaxel and temozolomide; 2-methylthiazolidine-2,4-dicarboxylic acid with paclitaxel and vincristine.

Especially preferred is a combination of 2-methylthiazolidine-2,4-dicarboxylic acid and/or salts of 2-methylthiazolidine-2,4-dicarboxylic acid with cisplatin, temozolomide and/or vincristine.

The combination preparation according to invention as well as the pharmaceutical formulation comprising 2-methylthiazolidine-2,4-dicarboxylic acid and the pharmaceutical formulation comprising at least one antiangiogenic and/ or cytotoxic and/or cytostatic compound may further contain at least one physiologically acceptable carrier, additive, auxiliary agent and/or solvent.

The combination preparation according to invention as well as the pharmaceutical formulations of the combination preparation according to the invention are prepared with conventional pharmaceutical practices with conventional solid or liquid carrier agents or diluents and with conventionally utilized pharmaceutical auxiliary agents suitably selected with respect to the intended pharmaceutical forms with a suitable dosage. Such pharmaceutical forms are for example tablets, film-coated tablets, layered tablets, sugar-coated tablets, capsules, micro capsules, micro pellets and pellets, pills, granulates, powders, powder blends, solutions, dispersions, suspensions, suppositories, emulsions, gels, ointments, syrups or prolonged release formulations or inhalation solutions or respectively powders. Moreover, according to invention the pharmaceutical compositions comprise formulations such as layered tablets for controlled and/or continuous release of the active agent as well as micro encapsulations as special pharmaceutical form.

Such compositions are for example suitable for inhalation or intravenous, intraperitoneal, intramuscular, subcutaneous, oral, rectal, transdermal, topical, intradermal, intragastric, intracutaneous, intravaginal, intranasal, intrabuccal, percutaneous or sublingual administration.

Especially advantageous forms of administration are oral and topical application, injection as well as inhalation.

Corresponding tablets can be obtained for example by mixing the compound which is applicable as intended by the invention and/or a salt thereof with known auxiliary agents for example inert diluents such as dextrose, sugar, sorbite, mannite, polyvinylpyrrolidone, disintegrants such as corn starch or alginic acid, binders such as starch or gelatine, lubricants such as magnesium stearate or talc and/or agents for achieving a prolonged release effect such as carboxypolymethylene, carboxymethyl cellulose, cellulose acetate phthalate or polyvinylacetate. The tablets can also consist of more layers.

Accordingly, sugar coated tablets can be prepared by coating of cores analogously prepared to the tablets with substances conventionally used in sugar-coatings, for example polyvinylpyrrolidone or shellac, gum arabicum, talc, titan dioxide or sugar. Thus, the film-coating can also consist of more layers, wherein in case of the tablets, the above mentioned auxiliary agents can be used.

Solutions or suspensions with the active agent of the invention may further contain sapidity agents such as saccharin, cyclamate or sugar as well as flavouring agents such as vanillin or orange extract. They may further contain suspending agents such as sodium carboxymethyl cellulose or preservatives such as p-hydroxybenzoates. Capsules containing active ingredients can be produced, for example, by mixing the active substance with an inert carrier such as lactose or sorbite, and by encapsulation thereof in gelatine capsules.

Suitable suppositories can be produced, for example, by mixing with the respective substrates such as neutral fats or polyethylene glycol or respectively with their derivatives.

Such formulations are for example suitable for inhalation or intravenous, intraperitoneal, intramuscular, subcutaneous, oral, rectal, transdermal, topical, intradermal, intragastric, intracutaneous, intravaginal, intranasal, intrabuccal, percutaneous or sublingual administration.

As pharmacologically acceptable carriers may be utilized for example lactose, starch, sorbite, sucrose, cellulose, magnesium stearate, dicalcium phosphate, calcium sulfate, talc, mannitol, ethyl alcohol and the like. Powders as well as tablets may consist of from about 5 to 95% of such a carrier.

As binders moreover starch, gelatine, natural sugars, natural and synthetic gums such as acacia gum or guar gum, sodium alginate, carboxymethyl cellulose, polyethylene glycol and waxes can be used. As lubricant boric acid, sodium benzoate, sodium acetate, sodium chloride and the like can be used.

Furthermore, disintegrants, coloring agents, flavoring agents and/or binders may be added to the pharmaceutical compositions.

Liquid formulations comprise solutions, suspensions, sprays and emulsions such as injection solutions based on water or water propylene glycol for parenteral injections.

For the preparation of suppositories, low-melting waxes, fatty acid esters and glycerides are preferably used.

Capsules are for example made of methyl cellulose, polyvinyl alcohols or denatured gelatines or starch.

As disintegrants can be used: starch, sodium carboxymethyl starch, natural and synthetic gums such as locust bean gum, karaya, guar, tragacanth and agar as well as cellulose derivatives such as methylcellulose, sodium carboxymethyl cellulose, microcrystalline cellulose as well as alginates, clays and bentonites. These components can be used in quantities ranging from 2 to 30% by weight.

As binders sugars, corn starch, rice or potatoes, natural gums such as acacia gum, gelatine, tragacanth, alginic acid, sodium alginate, ammonia calcium alginate, methylcellulose, sodium carboxymethyl cellulose, hydroxypropyl methylcellulose, polyvinylpyrrolidone as well as inorganic compounds such as magnesium aluminum silicate can be added. The binders can be added in quantities ranging from 1 to 30% by weight.

As lubricants can be used: stearates such as magnesium stearate, calcium stearate, potassium stearate, stearic acid, high-melting waxes as well as water soluble lubricants such as sodium chloride, sodium benzoate, sodium acetate, sodium oleate, polyethylene glycol and amino acids such as leucine. Such lubricants can be used in quantities ranging from 0.05 to 15% by weight.

The combination preparation according to invention is especially used in cancer therapy. It is not limited to particular carcinomas, tumor or cancer types. The indication is especially based on the type of the used antiangiogenic and/or cytotoxic and/or cytostatic compound, i.e. the cancer types where these antiangiogenic and/ or cytotoxic and/or cytostatic compound has been used so far as a single substance.

According to invention, the combination preparation can be used in case of the following tumors or respectively cancer types: acute and chronic myeloid leukemia, acute and chronic lymphatic leukemia, anal carcinoma, astrocytoma, basal cell carcinoma, small cell and non small cell bronchial carcinoma, Burkitt's lymphoma, CUP-syndrome, small intestine tumors, endometrial carcinoma, ependymoma, Ewing's tumors, gall bladder and bile duct carcinoma, glioblastoma, hairy cell leukemia, brain tumors (gliomas), brain metastases, testicle cancer, Hodgkin's disease, hypophysis tumors, carcinoids, Kaposi's sarcoma, laryngeal cancer, germ cell tumor, bone cancer, head and neck tumors, colon carcinoma, craniopharyngiomas, cancer in the mouth area and on the lip, liver cell carcinoma, liver metastases, eyelid tumor, stomach cancer, malignant melanoma, breast carcinoma, medulloblastomas, meningiomas, mycosis fungoides, neurinoma, renal cell carcinoma, Non-Hodgkin's lymphomas, oligodendroglioma, esophageal carcinoma, osteosarcoma, ovarian carcinoma, pancreatic carcinoma, penile carcinoma, plasmocytoma, prostate carcinoma, rectal carcinoma, retinoblastoma, thyroid carcinoma, spinalioma, thymoma, tube carcinoma, eye tumors, urethral cancer, urothelial carcinoma, vulva carcinoma, wart appearance, soft tissue tumors, Wilm's tumor, cervical carcinoma and tongue cancer.

### EXAMPLES

### Example 1

Examinations for the in vitro cytoxicity of 2-methylthiazolidine-2,4-dicarboxylic acid and N-acetylcysteine in human tumor cell lines were carried out.

Used Substances:

2-methylthiazolidine-2,4-dicarboxylic acid (MTDC), produced by Trommsdorff GmbH & Co. KG, Alsdorf (MTDC: 40 mg/ml in water dd),

N-acetylcysteine 40 mg/ml in water,

Temozolomide 5 mg/ml in DMSO

Cell Culture:

The cells were kept in culturing medium RPMI 1640 (Sigma, Munich) and in addition 10% FCS (Sigma, Munich) in 50 ml cell culture vials (Greiner) at 37° C. as well as under a 5% of CO₂ atmosphere.

Used Cell Lines:

Neuroglioma cells H4

Bladder carcinoma cells EJ

Cytotoxicity Assay:

Monolayers of the tumor cells were prepared by dispersion of 2×10⁶ tumor cells in 96-hole-plates (Greiner). After an incubation time of 24 hours the medium was replaced by a medium containing the test compounds. All assays were carried out four times.

After 72 h the quantity of living cells was determined by means of coloring with crystal violet.

For that purpose, the medium was removed from each hole in the plate, the plates were washed twice with PBS, the cells were fixed by means of PBS, containing 1% of paraformaldehyde and dried for 15 minutes at room temperature. Subsequently, the cells were rinsed with PBS and dried at room temperature.

Thereafter, the cells were colored with 1% crystal violet solution in water for two minutes, and after washing with water the optical density was determined by means of an ELISA spectrometer at 595 nm. The average values and standard deviations for the average values were determined for all four parallel tests.

Results:

In the human gliomas cells H4, 2-methylthiazolidine-2,4-dicarboxylic acid had a significant cytotoxic effect. An IC₅₀ value of 9.66 μg/ml was found. N-acetylcysteine up to comparatively high concentrations, however, had no effect.

Furthermore, the growth of the bladder carcinoma cell line EJ was not influenced by 2-methylthiazolidine-2,4-dicarboxylic acid.

When 2-methylthiazolidine-2,4-dicarboxylic acid, however, was administered together with the cytostatic temozolomide, a significantly increased sensitivity of the EJ cells to temozolomide compared to the tests with temozolomide in the presence of 2-methylthiazolidine-2,4-dicarboxylic acid was shown. The IC₅₀ value for temozolomide alone was of 1053.36 μg/ml. IN the presence of 200 μg/ml 2-methylthiazolidine-2,4-dicarboxylic acid, the IC₅₀ value dropped to 96.58 μg/ml.

In contrast, the presence of 1000 μg/ml N-acetylcysteine did not increase the effect of temozolomide on the EJ cells (IC₅₀ value of temozolomide alone: 1053.36 μg/ml; IC₅₀ value of temozolomide in the presence of 1000 μg/ml N-acetylcysteine: 903.24 μg/ml).

These test data prove the unexpected increase of the efficacy of combinations of a cytostatic with 2-methylthiazolidine-2,4-dicarboxylic acid compared to the administration of a single cytostatic and the non existent or low influence on the efficacy of a cytostatic or a cytotoxic or antiangiogenic substance by N-acetylcysteine.

### Example 2

Used Substances:

Sodium Salt of 2-methylthiazolidine-2,4-dicarboxylic acid (MTDC-Na) (Trommsdorff GmbH & Co. KG Arzneimittel);
Cisplatin (commercially available from Alfa);
Temozolomide (commercially available from Essex Pharma);
Vincristine (commercially available from Medac).

The Following Stock Solutions were Used:
MTDC-Na 40 mg/ml (dissolved in distilled water),
Cisplatin, 0.5 mg/ml (dissolved in distilled water),
Temozolomide, 5 mg/ml (dissolved in DMSO),
Vincristine 1 mg/ml (dissolved in distilled water).

The Following Cell Lines were Examined:
Neuroglioma H4
Bladder carcinoma EJ
Prostate carcinoma DU145
   Cell Cultures:

The cells were kept in culturing medium RPMI 1640 (Sigma, Munich) and in addition 10% FCS (Sigma, Munich) in 50 ml cell culture vials (Greiner) at 37° C. as well as under a 5% CO₂ atmosphere.

Cytotoxicity Assay:

Monolayers of the tumor cells were prepared by dispersion of 2×106 tumor cells in 96-hole-plates (Greiner).

After an incubation time of 30 hours (t=0) the medium was replaced by the medium containing the anti cancer preparation.

After an incubation time of another 24 hours (t=+24 h), the anti cancer agent was removed and the medium replaced.

MTDC-Na was added to the cells by means of an exchange of the present medium by a fresh medium containing MTDC-Na.

All of the tests were carried out three times.

The Following Concentrations of the Anti Cancer pPeparation were Us:

Temozolomide in a concentration of 0-1000 μg/ml,
Vincristine in a concentration of 0-200 ng/ml,
Cisplatin in a concentration of 0-25 μg/ml.

After 126 hours of incubation, the cells were collected and the quantity of living cells was determined by means of coloring with crystal violet.

For that purpose, the medium was removed, the hole plates were washed two times with PBS and afterwards the cells were fixed for 15 minutes at room temperature with a PBS-solution containing 1% paraformaldehyde. Thereafter, the plates were rinsed two times with PBS and dried at room temperature. The cells were colored for 2 minutes with a solution of 1% crystal violet in water.

After the washing with water the optical density was determined by means of an ELISA-Reader at 595 nm. The average values and the standard deviations of the average values were determined for the three test series.

IC₅₀-values were determined by means of plotting the active agent concentration versus the optical density.

The results are shown in table 1:

TABLE 1
Increase of the cytotoxic activity of MTDC-Na in case of a
combination with an other cytotoxic and/or cytostatic compound

| | | Increase of the activity |
|---|---|---|
| | Cytotoxic and/or cytostatic | Decrease of the IC₅₀ |
| Cell line | compound | value |
| Prostate carcinoma | Cisplatin/ | 2-fold |
| DU 145 | MTDC-Na (1000 μg/ml) | |
| Prostate carcinoma | Vincristine/ | 2.7-fold |
| DU 145 | MTDC-Na (1000 μg/ml) | |
| Bladder carcinoma | Cisplatin/ | 10-fold |
| EJ | MTDC-Na (1000 μg/ml) | |
| Bladder carcinoma | Temozolomide/ | 67-fold |
| EJ | MTDC-Na (1000 μg/ml) | |
| Bladder carcinoma | Vincristine/ | 6-fold |
| EJ | MTDC-Na (1000 μg/ml) | |
| Bladder carcinoma | Vincristine/ | 4.8-fold |
| EJ | MTDC-Na (1000 μg/ml) | |
| Neuroglioma | Cisplatin/ | 4-fold |
| H4 | MTDC-Na (200 μg/ml) | |
| Neuroglioma | Temozolomide/ | 15-fold |
| H4 | MTDC-Na (200 μg/ml) | |

1. Combination preparations comprising 2-methylthiazolidine-2,4-dicarboxylic acid and/or physiologically acceptable salts thereof and at least one cytotoxic and/or cytostatic compound.

2. Combination preparation according to claim 1 comprising a pharmaceutical formulation containing 2-methylthiazolidine-2,4-dicarboxylic acid and/or physiologically acceptable salts thereof as well as at least one other pharmaceutical formulation containing the at least one antiangiogenic and/or cytotoxic and/or cytostatic compound.

3. Combination preparation according to claim 1 further comprising at least one physiologically acceptable carrier, additive, auxiliary agent and/or solvent.

4. Combination preparation according to claim 2, wherein the antiangiogenic and/ or cytotoxic and/or cytostatic compounds can be selected from the classes comprising alkylating agents, antibiotics with cytostatic properties, antimetabolites, microtubule inhibitors, topoisomerase inhibitors, compounds containing platinum, alkaloids, podophyllotoxins, taxanes, hormones, immunomodulators, monoclonal antibodies, signal transductors (signal transduction molecules) and cytokines.

5. Combination preparation according to claim 4, wherein the alkylating agents, antibiotics with cytostatic properties, antimetabolites, microtubule inhibitors, topoisomerase inhibitors, compounds containing platinum, alkaloids, podophyllotoxins, taxanes, hormones, immunomodulators, monoclonal antibodies, signal transductors (signal transduction molecules) and cytokines are selected from the group comprising chlorethamine, cyclophosphamide, trofosfamide, ifosfamide, melphalan, chlorambucil, busulfan, thiotepa, carmustine, lomustine, dacarbazine, procarbazine, temozolomide, treosulfan, estramustine, nimustine, daunorubicin as well as liposomal daunorubicin, doxorubicin, adriamycin as well as liposomal adriamycin, dactinomycin, mitomycin C, bleomycin, epirubicin (4-epi-adriamycin), idarubicin, dactinomycin, mitoxantrone, mitomycin C, plicamycin, amsacrine, actinomycin D, methotrexate, 5-fluorouracil, 6-thioguanine, 6-mercaptopurine, fludarabine, cladribine, pentostatin, gemcitabine, cytarabine, azathioprine, raltitrexed, capecitabine, cytosine arabinoside, thioguanine, mercaptopurine, vincristine, vinblastine, vindesine, etoposide, teniposide, cisplatin, carboplatin, oxaliplatin, vinea alkaloids, vinorelbine, etoposide, teniposide, camptothecin, topotecan, irinotecan, paclitaxel, docetaxel, hydroxycarbamide (hydroxyurea), imatinib, miltefosine, amsacrine, topotecan (topoisomerase-I inhibitor), pentostatin, bexarotene, tretinoin, asparaginase, trastuzumab, alemtuzumab, rituximab, glucocorticoids (prednisone), estrogens (fosfestrol, estramustine), LHRH (buserelin, goserelin, leuprorelin, triptorelin), flutamide, cyproterone acetate, tamoxifen, toremifen, aminoglutethimide, formestane, exemestane, letrozole, anastrozole, interleukin-2, interferon-α, erythropoietin, G-CSF, trastuzumab, rituximab, gefitinib, ibritumomab, levamisole as well as retinoids.

6. Combination preparation according to claim 5, wherein alkylating agents, antibiotics with cytostatic properties, antimetabolites, microtubule inhibitors, topoisomerase inhibitors, compounds containing platinum, alkaloids, podophyllotoxins, taxanes, hormones, immunomodulators, monoclonal antibodies, signal transductors (signal transduction molecules) and cytokines are selected from the group comprising cisplatin, temozolomide and vincristine.

7. Use of the combination preparation according to claim 1 for prophylaxis and/or treatment of tumors and cancer.

8. Use according to claim 7, wherein the tumors and cancers concerned are acute and chronic mycloid leukemia, acute and chronic lymphatic leukemia, anal carcinoma, astrocytoma, basal cell carcinoma, small cell and non small cell bronchial carcinoma, Burkitt's lymphoma, CUP-syndrome, small intestine tumors, endometrial carcinoma, ependymoma, Ewing's tumors, gall bladder and bile duct carcinoma, glioblastoma, hairy cell leukemia, brain tumors (gliomas), brain metastases, testicle cancer, Hodgkin's disease, hypophysis tumors, carcinoids, Kaposi's sarcoma, laryngeal cancer, germ cell tumor, bone cancer, head and neck tumors, colon carcinoma, craniopharyngiomas, cancer in the mouth area and on the lip, liver cell carcinoma, liver metastases, eyelid tumor, stomach cancer, malignant melanoma, breast carcinoma, medulloblastomas, meningiomas, mycosis fungoides, neurinoma, renal cell carcinoma, Non-Hodgkin's lymphomas, oligodendroglioma, esophageal carcinoma, osteosarcoma, ovarian carcinoma, pancreatic carcinoma, penile carcinoma, plasmocytoma, prostate carcinoma, rectal carcinoma, retinoblastoma, thyroid carcinoma, spinalioma, thymoma, tube carcinoma, eye tumors, urethral cancer, urothelial carcinoma, vulva carcinoma, wart appearance, soft tissue tumors, Wilm's tumor, cervical carcinoma and tongue cancer.

9. Use of 2-methylthiazolidine-2,4-dicarboxylic acid and/or physiologically acceptable salts for increasing the efficacy of cytostatics.

10. Use according to claim 9, wherein the cytostatics are selected from the group comprising alkylating agents, antibiotics with cytostatic properties, antimetabolites, microtubule inhibitors, topoisomerase inhibitors, compounds containing platinum, alkaloids, podophyllotoxins, taxanes, hormones, immunomodulators, monoclonal antibodies, signal transductors (signal transduction molecules) and cytokines.

11. Use according to claim 10, wherein the alkylating agents, antibiotics with cytostatic properties, antimetabolites, microtubule inhibitors, topoisomerase inhibitors, compounds containing platinum, alkaloids, podophyllotoxins, taxanes, hormones, immunomodulators, monoclonal antibodies, signal transductors (signal transduction molecules) and cytokines are selected from the group comprising chlorethamine, cyclophosphamide, trofosfamide, ifosfamide, melphalan, chlorambucil, busulfan, thiotepa, carmustine, lomustine, dacarbazine, procarbazine, temozolomide, treosulfan, estramustine, nimustine, daunorubicin as well as liposomal daunorubicin, doxorubicin (adriamycin) as well as liposomal adriamycin, dactinomycin, mitomycin C, bleomycin, epirubicin (4-epi-adriamycin), idarubicin, dactinomycin, mitoxantrone, plicamycin, amsacrine, actinomycin D, methotrexate, 5-fluorouracil, 6-thioguanine, 6-mercaptopurine, fludarabine, cladribine, pentostatin, gemcitabine, cytarabine, azathioprine, raltitrexed, capecitabine, cytosine arabinoside, thioguanine, mercaptopurine, vincristine, vinblastine, vindesine, etoposide, teniposide, cisplatin, carboplatin, oxaliplatin, vinca alkaloids, vinorelbine, teniposide, camptothecin, topotecan, irinotecan, paclitaxel, docetaxel, hydroxycarbamide (hydroxyurea), imatinib, Miltefosine®, amsacrine, topotecan (topoisomerase-I inhibitor), pentostatin, bexarotene, tretinoin, asparaginase, trastuzumab, alemtuzumab, rituximab, glucocorticoids (prednisone), estrogens (fosfestrol, estramustine), LHRH (buserelin, goserelin, leuprorelin, triptorelin), flutamide, cyproterone acetate, tamoxifen, toremifen, aminoglutethimide, formestane, exemestane, letrozole, anastrozole, interleukin-2, interferon-α, erythropoietin, G-CSF, trastuzumab, rituximab, gefitinib, ibritumomab, levamisole as well as retinoids.
